(19) United States
(12) Patent Application Publication
(10) Pub. No.: US 2024/0079109 A1
(43) Pub. Date: Mar. 7, 2024

(54) PHARMACOLOGICAL RECOMMENDATION SYSTEM

(71) Applicant: POSOS, Amiens (FR)

(72) Inventors: Emmanuel BILBAULT, Amiens (FR); Benjamin GRELIE, Amiens (FR); Pierre DEMBIERMONT, Amiens (FR); Pierre LUCE, Amiens (FR)

(73) Assignee: POSOS, Amiens (FR)

(21) Appl. No.: 18/003,329

(22) PCT Filed: Jun. 24, 2021
(86) PCT No.: PCT/FR2021/051167

§ 371 Date: Jun. 15, 2023

(30) Foreign Application Priority Data
Jun. 26, 2020 (FR) 2006761

(51) Int. Cl. G16H 20/10; G16H 10/60

(52) U.S. Cl. CPC G16H 20/10; G16H 10/60

(57) ABSTRACT

A pharmacological recommendation system comprising a memory (4) receiving a database comprising context entries associated with a treatment indication, a side effect or a patient profile, and drug entries wherein each drug entry combines on the one hand drug data comprising a generic drug name, a drug name, dosing information and pharmaceutical information, and on the other hand constraint data comprising a list of therapeutic indications, a list of side effects, a list of contraindicated association entries and a list of contraindicated patient profiles, the drug entries and the context entries in the database being linked together by a graph, an analyser (8) designed to receive data entries each identifying drug data comprising at least one generic drug name or a drug name and context entries comprising at least one treatment indication, a side effect or a patient profile, and to determine on each occasion a drug entry or a context entry, and a recommendation engine (10) designed to perform a search in the graph based on drug entries and context entries determined by the analyser (8), and to deliver pharmacological recommendation data comprising one or more from—matching data determined based on an overlap between the constraint data defined by the drug entries and the context entries determined by the analyser (8), —alternative drug entry data for one or more of the drug entries determined by the analyser (8), which alternative drug entries have constraint data that comprise a list of treatment indications compatible with the treatment indication(s) in the context entries determined by the analyser (8) and/or the list of treatment indications in the drug entries for which they constitute an alternative, a list of side effects and/or a list of contraindicated patient profiles compatible with the side effects and/or the patient profiles in the context entries determined by the analyser (8), for which the lists of contraindicated association entries do not overlap with each other or with the other drug entries determined by the analyser (8).

The invention relates to the field of pharmacology, and in particular drug recommendation.

When a doctor prepares a prescription for a patient, they must make sure that the prescribed drugs are adapted to the clinical situation of their patient. Indeed, it frequently happens that the prescription contains a plurality of drugs. As not all drugs are compatible with one another, the doctor must therefore check that there is no risk of drug interaction. Even when the prescription must only contain a single drug, the doctor must check that it is compatible with the pathophysiological patient profile of the patient for which they are prescribing it.

The doctor must also make sure that the prescribed drug(s) do not have contraindications for their patient. In addition, the tolerance for the prescribed treatment(s) is variable from one patient to another, the doctor must regularly modify their prescription to limit its iatrogenicity. Finally, supply shortages are common, and the doctor has to find alternatives on a daily basis to the prescribed treatments. They must then find drugs having the same treatment indications and carry out their prescription safety process again.

All of these checks make the prescribing exercise complex and dangerous, because they represent significant sources of error. What is more, the efforts dedicated to this exercise prevent doctors from dedicating all their energy to questions relating directly to their patients. Furthermore, the amount of data relating to the drugs is constantly growing, which makes these problems increasingly important.

Historically, doctors used thick encyclopaedias such as the Vidal (registered trademark) to carry out these searches. The latter contained all the drugs approved by the health authorities, under their trade name. Each entry comprised a description of the drug, its composition (in active ingredient(s) and in excipient(s), the active ingredient dose (or equivalent in active ingredient base), its pharmaceutical form, a description of the treatment indications, side effects, contraindicated patient profiles, as well as contraindicated drug combinations.

Over time, these encyclopaedias have been digitised, and are now accessible online, for example under the name of Claude Bernard (registered trademark), Thériaque (registered trademark), or also Vidal (registered trademark) database. However, these solutions are only a simple digitising of the encyclopaedias: they propose easy access to documentation known in the medical literature or the search for predefined elements to identify the problems linked to the prescription (drug interactions, contraindications, particular monitoring).

It is not possible to search for information in addition to that predefined, and no solution/alternative is proposed. In addition, as the information is not standardised, it is also not possible to systematise the processing of the information. Indeed, all it takes is for a side effect or a treatment indication to be incorrectly entered or entered in two equivalents, but distinct, ways, and a portion of the information is irremediably lost.

Therefore, the user must carry out a plurality of searches within a plurality of drug datasheets to obtain the information: presence of side effect, contraindication for a given patient profile, interaction with the drug class. Furthermore, the existence of a drug having the same indication without this side effect is information that is not available in any encyclopaedia or any tool: each drug of an indication should be reviewed then it should be sought whether the undesirable effect concerned is mentioned. In addition, when the doctor must manage a plurality of drugs, the situation becomes even more complex. Finally, it is impossible to question these solutions in natural language, or at least other than according to the strict formalism of these solutions. Efforts have been deployed in order to standardise the drug datasheets. This is for example the case of the French patent FR 3 059 118. This standardisation makes it possible to enter a prescription on an interface, the age of the patient, as well as pathophysiological features (such as pregnancy, renal or hepatic insufficiency, etc.), and to detect interactions and associated undesirable effects. Complementary information relating to the pathophysiological patient profile of the patient may also be proposed, the associated publications, the STOPP criteria details and the shared drug review may be published.

However, in practice this system only standardises the active ingredients. All other information remains “dead”, such as in known encyclopaedias. Thus, the resulting database offer a fairly limited use in medical practice.

The invention improves the situation. To this end, it proposes a pharmacological recommendation system, comprising a memory receiving a database comprising context entries associated with a treatment indication, a side effect or a patient profile, and drug entries wherein each drug entry combines on the one hand drug data comprising a generic drug name, a drug name, dosing data and pharmaceutical data, and on the other hand constraint data comprising a list of treatment indications, a list of side effects, a list of contraindicated association entries, a list of contraindicated patient profiles, the drug entries and the context entries of the database being linked together by a graph, an analyser arranged to receive data entries each designating drug data comprising at least one generic drug name or a drug name and context data comprising at least one treatment indication, a side effect or a patient profile, and to determine in each case a drug entry or a context entry, and a recommendation engine arranged to perform a search in the graph based on drug entries and context entries determined by the analyser, and to return pharmacological recommendation data comprising one or more from
matching information determined based on the overlap between the constraint data defined by the drug entries and the context entries determined by the analyser,
alternative drug entry information for one or more of the drug entries determined by the analyser, which alternative drug entries have constraint data that comprise a list of treatment indications compatible with the treatment indication(s) of the context entries determined by the analyser and/or the list of treatment indications of the drug entries for which they constitute the alternative, a list of side effects and/or a list of contraindicated patient profiles compatible with the side effects and/or the patient profiles of the context entries determined by the analyser, for which the lists of contraindicated association entries do not overlap with each other or with the other drug entries determined by the analyser.

This device is particularly advantageous because it makes it possible for doctors to use in a much more efficient way the information relating to the drugs. In particular, it makes it possible to propose treatment alternatives, while making it possible for the user to enter information freely.

According to miscellaneous embodiments, this system may have one or more of the following features:
when the analyser determines a generic drug name, it returns the drug entries which drug data comprise this generic drug name,
the system further comprises a user interface to enter said entry data designating each of the drug data and/or context data,
the analyser is arranged to implement a NERL-type recognition engine to determine on each occasion a drug entry or a context entry based on said entry data designating each of the drug data and/or context data,
the analyser comprises an LSTM neural network, and a CRF layer to implement the NERL-type recognition engine, and
the memory receives drug entries wherein the drug data further comprise ingredient data, and/or the constraint data comprise specific data such as one or more age, weight and/or pathophysiological patient profile groups.

The invention also relates to a pharmacological recommendation method implemented by computer, comprising the following operations:
a) Providing a database comprising context entries associated with a treatment indication, a side effect or a patient profile, and drug entries wherein each drug entry combines on the one hand drug data comprising a generic drug name, a drug name, dosing data and pharmaceutical data, and on the other hand constraint data comprising a list of treatment indications, a list of side effects, a list of contraindicated association entries, a list of contraindicated patient profiles, the drug entries and the context entries of the database being linked together by a graph,
b) Receiving data entries designating each of the drug data comprising at least one generic drug name or a drug name and context data comprising at least one treatment indication, a side effect or a patient profile,
c) For each data of operation b), determining on each occasion a drug entry or a context entry,
d) Searching the graph of the database of operation a) based on the drug entries and context entries of operation c), and returning pharmacological recommendation data comprising one or more from
matching information determined based on the overlap between the constraint data defined by the drug entries and the context entries of operation c),
alternative drug entry information for one or more drug entries of operation c), which alternative drug entries have constraint data that comprise a list of treatment indications compatible with the treatment indication(s) of the context entries of operation c) and/or the list of treatment indications of the drug entries for which they constitute the alternative, a list of side effects and/or a list of contraindicated patient profiles compatible with the side effects and/or the patient profiles of the context entries of operation c), for which the lists of contraindicated association entries do not overlap with each other or with the other drug entries of operation c).

According to miscellaneous embodiments, this method may have one or more of the following features:
when operation c) determines a generic drug name, all of the drug entries which drug data of comprise this generic drug name are returned,
operation c) comprises implementing an NERL-type recognition engine to determine on each occasion a drug entry or a context entry based on said entry data designating each of the drug data and/or context data,
operation c) comprises implementing an NERL-type recognition engine comprising an LSTM neural network, and a CRF layer,
operation b) is implemented by means of a user interface, and/or by access to data of other sources, in particular by access to the Patient Medical File, and
the drug entries wherein the drug data further comprise ingredient data, and/or the constraint data comprise specific data such as one or more age, weight and/or pathophysiological patient profile groups.

Finally, the invention relates to a computer program product comprising instructions for implementing the method when it is executed on a computer, and a storage medium wherein the computer program product is saved.

Other features and advantages of the invention will become more apparent upon reading the following description, taken by way of illustrative and non-limiting examples, taken from drawings wherein:

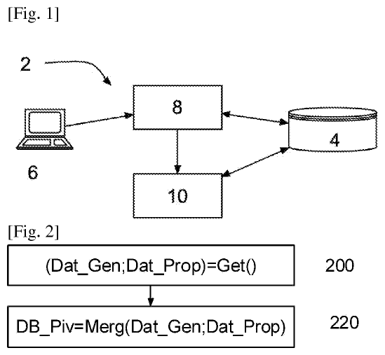

FIG. 1 shows a schematic example of a system according to the invention, and

FIG. 2 shows an example of implementation making it possible to obtain the database of the system of FIG. 1.

The drawings and the following description mainly contain elements of certain character. Therefore, not only may they be used to better understand the present invention, but also to help to define it, if necessary.

FIG. 1 shows a schematic example of implementation of the invention. In this example, the system 2 comprises a memory 4, a user interface 6, an analyser 8 and a recommendation engine 10.

The memory 4 may be any type of specific data storage for receiving digital data: hard drive, flash memory hard drive flash memory in any form, random access memory, magnetic disk, storage distributed locally or in the cloud, etc. The data calculated by the device may be stored on any type of memory similar to the memory 4, or on the latter. These data may be erased after the device has carried out its tasks or kept.

In the example described here, the memory 4 receives a drugs database. This database, specific to the invention, is particular in that it constitutes the first database wherein all of the critical description information of a drug, that is to say not only its generic drug name (also called INN for International Nonproprietary Name), but also its treatment indications, its side effects, the contraindicated patient profiles, and the combinations with other contraindicated drugs have all been processed, standardised and formatted in order to generate a truly pivot database. As explained in the introduction of the application, this concerns the first database of this type, the best efforts to date only having at best brought together the INN and the drug interactions, because this information contains almost no ambiguity. Concerning side effects and contraindicated patient profiles, no database makes it possible to bring together identical side effects or identical contraindicated patient profiles but expressed with different terms. Likewise, certain treatment indications may overlap or be very similar, but no database, until that of the invention has made it possible to actually capture these relationships.

To this end, FIG. 2 shows an example of the method implemented to obtain the database of the invention. As can be seen in this figure, in one operation 200, generic data Dat_Gen and proprietary data Dat_Prop are obtained by executing a Get( ) function. The Get( ) function is in the example described here a function that accesses locations wherein the generic data and the proprietary data are stored.

The generic data Dat_Gen may for example be obtained by accessing miscellaneous data sources on the drugs, such as the Thériaque database discussed above, the ANSM (Agence nationale de sécurité du médicament et des produits de sante—National Agency for the Safety of Medicines and Health Products) database, one or more ontology databases such as CIM-10, ATC, CISMEF, or SNOMED-CT, etc.

The proprietary data Dat_Prop are obtained based on annotated data, for example to establish equivalences between terms designating side effects, patient profiles or treatment effects. These data have, in the example described here, been generated by the Applicant.

Subsequently, in one operation 220, a Merg( ) function receives the generic data Dat_Gen and the proprietary data Dat_Prop in order to standardise the generic data, then format them in order to make them usable within the scope of the invention. For example, this formatting may include preparing fields intended to be returned directly in the JSON format used by the system 2. Generally, this formatting may be used in order to duplicate some of the organised data of the database in a “ready-to-use” form when a search is formulated.

As will be seen below, the data of the pivot database may be seen as having two natures:

Drug entries, which contain information that relates to each drug,

Context entries, which contain general categories such as the treatment indications, the side effects, the patient profiles, particular populations, etc.

The drug entries themselves combine two types of data:

On the one hand drug data, which relate directly to the drug, such as a generic drug name, a drug name, the name of the active ingredient and dosing data and pharmaceutical data,

On the other hand, constraint data that are associated with this drug, such as a list of treatment indications, a list of side effects, a list of contraindicated association entries, a list of contraindicated patient profiles.

The constraint data constitute a combination of a drug entry with one or more context entries and/or other drug entries. In some advantageous embodiments, the drug data may comprise ingredient data constituting the drug, and/or the constraint data may also comprise specific data, such as age, weight, or pathophysiological patient profile groups for recognising and combining the ontological subtleties specific to each drug for these specific data.

In the example described here, the ontological conflicts and disambiguation are resolved by systematic expert arbitrations and the use of biomedical embeddings: a statistic model, based on the fastText method described in the article by Bojanowski and al. “Fasttext.Zip: Compressing Text Classification Models”, 2016, arXiv:1612.03651, trained on proprietary data, embeds the medical vocabulary into a vector space that preserves the semantic similarity. Each set of words may then be associated with one or more representatives in an ontology.

As output, the Merg( ) function returns the pivot database wherein all of the information has been organised as described above.

It goes without saying that the preceding may be performed by portions. For example, it is not necessary to fully reconstruct the pivot database every time, and the execution of the function of FIG. 2 may make it possible for example to only update the entries that need to be in view of changes in the generic data or in the proprietary data, or in the Thériaque database and/or the ontologies.

Conversely, it may also be provided to entirely regenerate the database at regular intervals, in order to offer a “new” database at regular intervals.

The user interface 6 may be any human machine interface making it possible for a user (for example a doctor) of the system 2 to enter a search. This search may particularly advantageously take the form of textual information in natural language, and the user interface 6 may also evaluate it in real time, in order to make the predictive proposal in the form of filling suggestions. For example, if a user types the expression “Para” in a drug name search field, the user interface 6 may automatically propose to them to introduce the words “Paracetamol”, or “Paraffin”, etc. The user interface 6 also proposes medical entities, taken from a database approved by the HAS, semantically similar to the expression entered by the user, thanks to medical embeddings. For example, if a user types the word “fatigue”, the user interface 6 proposes the word “asthenia”. The user interface 6 may therefore take any useful form, regardless of a keyboard, a stylus, a microphone linked to voice recognition, etc. The entry of these data by the user may also be performed by filling in a plurality of fields provided for this purpose, such as a “Drug” field and a “Context” field. In the “Drug” field, a user may enter a drug trade name (for example Advil, Dafalgan, etc.), or a generic drug name (for example paracetamol or ibuprofen). In the “Constraints” field, the user may enter the topics wherein they seek to constrain their drug search. Thus, this is where they will enter the side effects that they seek to avoid, for example solar urticaria, the patient profile of a particular patient in order to avoid a contraindication, for example “renal insufficiency”, or also a treatment indication that they seek to treat.

The analyser 8 and the recommendation engine 10 are elements directly or indirectly accessing the memory 4. They may be produced in the form of an appropriate computer code executed on one or more processors. By processors, it must be understood any processor adapted to the calculations described below. Such a processor may be produced in any known way, in the form of a microprocessor for personal computer, of an FPGA or SoC type dedicated chip, of a calculation resource on a grid or in the cloud, of a microcontroller, or of any other specific form to provide the calculation power necessary for the embodiment described below. One or more of these elements may also be produced in the form of specialist electronic circuits such as an ASIC. A combination of processor and of electronic circuits may also be envisaged.

In the example described here, the purpose of the analyser 8 is to determine the elements of a user search based on the entry received from the user interface 6. Thus, a user may write a phrase, for example “headache treatment by ibuprofen”, and the analyser 8 will automatically identify, a treatment indication “Migraine” and a generic drug name “ibuprofen”. As seen above, the analyser 8 may also directly receive text chains associated with a drug and/or with a context. The analyser 8 may also receive data from other sources (for example the Patient Medical File, the hospital and prescription assistance software), for example concomitant treatments, the patient profile of the patient with their diagnosis, their age, their weight, etc.

In the example described here, the analyser 8 implements an NERL (Named Entity Recognition and Linking) type recognition engine created by the Applicant. This engine is based on an LSTM (long short-term memory) neural network, followed by a CRF (Conditional Random Field) layer, as in the scientific article by Lample and al “Neural architectures for named entity recognition”, 2016, arXiv: 1603.01360. This engine is trained based on 100,000 manually annotated extracts in the IOB (Inside-outside-beginning) format and makes it possible to detect and find in a text the name(s) of related drugs, or other medical entities such as pathologies, symptoms, treatment classes, doses, etc., by word embedding. In practice, the NERL-type recognition engine will generate similar embeddings for similar medical terms. For example, asthenia and fatigue will have similar embeddings, which will therefore link them to one another in the searches of the database.

Thus, the combination of the analyser 8 with the pioneer database of the invention makes it possible to actually use all of the potential unexploited up to now. Indeed, the ambiguity of notions exists for encyclopaedias as well as for users (for example doctors). Consequently, the pivot database alone would be insufficient because it would ultimately correspond to imposing an ontological vision to doctors. Conversely, the analyser 8 alone would also be insufficient, because it would not be capable of adapting to the subtleties of the ontologies.

However, the combination of the two makes it possible for a doctor to express their search and effectively access the content of the drug encyclopaedias, as well as exhaustively cover and question the medical information linked to the drug in question, which was until now impossible with the aid of the systems developed to date.

What is more, the annotation of scientific documents and the structuring of raw data into proprietary ontologies to constitute the database make it possible to generate searchable data having a richness to date unknown. For example, this database may contain specific data such as the age, the weight, the pathophysiological patient profiles to recognise and associate the ontological subtleties specific to each drug for these specific data.

Subsequently, these data may be used in order to constitute the NERL-type recognition engine. This makes it possible for users to enter information specific to the patients listed above in natural language, and to access in return generic ontologies and propose the adequate dosing, and/or the drug alternatives and/or the drug interactions depending on the constraints identified by the NERL-type recognition engine.

Thus, it becomes for example possible for a user to obtain the optimal dosing of a drug for a patient according to their age (differentiations according to the various dosing intervals), their weight, or also comorbidities. Likewise, the structuring of pathophysiological data into proprietary ontologies, such as the various ages, weights, and the contraindicated patient profiles (for example hepatic insufficiency, etc.) makes it possible for users to access treatment alternatives or other medical information according to the specificities of the patients.

This is performed by the recommendation engine 10, which makes it possible to search the pivot database based on information entered by the user and removed by the analyser 8.

Thus, the recommendation engine 10 makes it possible to implement all use cases that such a system shows.

For example, a database search may be to obtain the drugs making it possible to treat a treatment indication. For this, the user may for example only enter the treatment indication. This search may be made more subtle by specifying in addition the side effects to avoid, or a patient profile of a patient.

Where the invention takes on all of its potential, is that it becomes possible to immediately obtain the list of drugs known to cause one or more given undesirable effect(s); adding a treatment indication could trigger a function that identifies the other drugs that treat this pathology, that is to say the treatment alternatives, and to only propose those that do not cause the side effect(s) mentioned in the search. Adding one or more pathophysiological patient profiles and concomitant drugs will make it possible to further refine this list of treatment alternatives. Likewise, this search may be enriched over time in order to add the various drugs that a patient takes already or that a doctor envisages prescribing.

Yet another variant will be the recommendation of alternatives. Indeed, when the recommendation engine 10 identifies an incompatibility between two drugs, or a side effect or a contraindicated patient profile, it may also automatically search for an alternative drug that treats the treatment indication of a drug designated by the user and that has no risk. In an even more sophisticated variant, the recommendation engine 10 may automatically study all alternative options in the event of a drug contraindication, by seeking to replace one then the other of the drugs.

The recommendation engine 10 may therefore issue recommendations and integrate them into a graphic interface by means of data in the JSON format that it recovers from the pivot database based on its searches.

These features are advantageous, because usually 10 to 15 minutes are needed to find the alternatives to a drug, and to check that they are compatible with the current treatments, that they are not contraindicated with the pathophysiological patient profile of the patient, and that they do not cause a side effect in particular. Added to this are the risks of errors that the invention makes it possible to avoid, and the fact that the user may focus better on the advice to be given to their patient, and less on what could be qualified as housekeeping.

It appears from the preceding that the system 2 is particularly adapted to a client/server type implementation, for example a website that a doctor logs into and introduces the data of their search, then the analyser 8 and the recommendation engine 10 on the server side send back to them the relevant information. Nevertheless, it goes without saying that other implementations are possible, integrally on a single machine, or other. In addition, the invention will also integrate naturally into drug delivery or prescription software, as well as within hospital software.

1. Pharmacological recommendation system, comprising a memory (4) receiving a database comprising context entries associated with a treatment indication, a side effect or a patient profile, and drug entries wherein each drug entry combines on the one hand drug data comprising a generic drug name, a drug name, dosing data and pharmaceutical data, and on the other hand constraint data comprising a list of treatment indications, a list of side effects, a list of contraindicated association entries, a list of contraindicated patient profiles, the drug entries and the context entries of the database being linked together by a graph, an analyser (8) arranged to receive data entries each designating drug data comprising at least one generic drug name or a drug name and context data comprising at least one treatment indication, a side effect or a patient profile, and to determine on each occasion a drug entry or a context entry, and a recommendation engine (10) arranged to perform a search in the graph based on drug entries and context entries determined by the analyser (8), and to return pharmacological recommendation data comprising one or more among
matching information determined based on the overlap between the constraint data defined by the drug entries and the context entries determined by the analyser (8),
alternative drug entry information for one or more of the drug entries determined by the analyser (8), which alternative drug entries have constraint data which comprise a list of treatment indications compatible with the treatment indication(s) of the context entries determined by the analyser (8) and/or the list of treatment indications of the drug entries for which they constitute the alternative, a list of side effects and/or a list of contraindicated patient profiles compatible with the side effects and/or the patient profiles of the context entries determined by the analyser (8), for which the lists of contraindicated association entries do not overlap with each other or with the other drug entries determined by the analyser (8).

2. Recommendation system according to claim 1 wherein, when the analyser (8) determines a generic drug name, it returns the drug entries which drug data comprise this generic drug name.

3. Recommendation system according to claim 1 or 2, further comprising a user interface (6) for inputting said entry data designating each of the drug data and/or context data.

4. System according to one of the preceding claims, wherein the analyser (8) is arranged to implement a NERL-type recognition engine to determine each time a drug entry or a context entry based on said entry data designating each of the drug data and/or context data.

5. System according to claim 4, wherein the analyser (8) comprises an LSTM (long short-term memory) neural network, and a CRF (Conditional Random Field) layer to implement the NERL-type recognition system.

6. System according to one of the preceding claims, wherein the memory (4) receives drug entries in which the drug data further comprise ingredient data, and/or the constraint data comprise specific data such as one or more age, weight and/or pathophysiological patient profile groups.

7. Pharmacological recommendation method implemented by computer, comprising the following operations:
a) Providing a database comprising context entries associated with a treatment indication, a side effect or a patient profile, and drug entries wherein each drug entry combines on the one hand drug data comprising a generic drug name, a drug name, dosing data and pharmaceutical data, and on the other hand constraint data comprising a list of treatment indications, a list of side effects, a list of contraindicated association entries, a list of contraindicated patient profiles, the drug entries and the context entries of the database being linked together by a graph,
b) Receiving data entries designating each of the drug data comprising at least one generic drug name or a drug name and context data comprising at least one treatment indication, a side effect, or a patient profile,
c) For each data of operation b), determining each time a drug entry or a context entry,
d) Searching the graph of the database of operation a) based on the drug entries and context entries of operation c), and returning pharmacological recommendation data comprising one or more from
matching information determined based on the overlap between the constraint data defined by the drug entries and the context entries of operation c),
alternative drug entry information for one or more drug entries of operation c), which alternative drug entries have constraint data that comprise
a list of treatment indications compatible with the treatment indication(s) of the context entries of operation c) and/or the list of treatment indications of the drug entries for which they constitute the alternative,
a list of side effects and/or a list of contraindicated patient profiles compatible with the side effects and/or the patient profiles of the context entries of operation c), for which the lists of contraindicated association entries do not overlap with each other or with the other drug entries of operation c).

8. Method according to claim 7, wherein, when operation c) determines a generic drug name, all of the drug entries which drug data comprise this generic drug name are returned.

9. Method according to claim 7 or 8, wherein operation c) comprises implementing a NERL-type recognition engine to determine each time a drug entry or a context entry based on entry data designating each of the drug data and/or context data.

10. Method according to claim 9, wherein operation c) comprises implementing a NERL-type recognition engine comprising an LSTM (long short-term memory) neural network, and a CRF (Conditional Random Field) layer.

11. Method according to one of the preceding claims, wherein operation b) is implemented by means of a user interface, and/or by access to data of other sources, in particular by access to the Patient Medical File.

12. Method according to one of the preceding claims, wherein the drug entries wherein the drug data further comprise ingredient data, and/or the constraint data comprise specific data such as one or more age, weight and/or pathophysiological patient profile groups.

13. Computer program product comprising instructions for implementing the method according to one of claims 7 to 12 when it is executed on a computer.

14. Storage medium wherein the computer program product is saved according to claim 13.
